(19) United States
(12) Patent Application Publication
(10) Pub. No.: US 2013/0096436 A1
(43) Pub. Date: Apr. 18, 2013

(54) INDUCTIVE TUNING SYSTEM FOR ULTRASOUND TRANSDUCER

(71) Applicants: Blake Little, Bothell, WA (US); Michael Greaves, Kirkland, WA (US)

(72) Inventors: Blake Little, Bothell, WA (US); Michael Greaves, Kirkland, WA (US)

(73) Assignee: MEDICIS TECHNOLOGIES CORPORATION, Bothell, WA (US)

(21) Appl. No.: 13/275,192

(22) Filed: Oct. 17, 2011

(51) Int. Cl. A61B 8/14; H05K 13/00; H01R 43/00

(52) U.S. Cl. 600/459; 29/825; 29/593

(57) ABSTRACT

Medical devices are disclosed that include a tunable ultrasonic transducer assembly, as well as related assemblies and methods. A medical device includes a transducer controller, a transducer having an input side connected with the ultrasound transducer controller via an input electrical conductor, and a tuning assembly that is electrically connected between the input electrical conductor and the ground potential. The tuning assembly is adjustable to provide a selected inductance from a number of different inductance values. The tuning assembly includes a multi-position switch and a plurality of tuning inductors. The switch is operable to select combinations of tuning inductors, thereby providing a selected inductance value between the input electrical connector and the ground potential so as inductively tune the medical device.

### BACKGROUND

Ultrasonic medical systems, such as ultrasonic diagnostic imaging systems and ultrasonic therapeutic systems, are in widespread use. Ultrasonic diagnostic imaging systems are used to perform ultrasonic imaging and measurements. For example, cardiologists, radiologists, and obstetricians use ultrasonic imaging systems to examine the heart, various abdominal organs, or a developing fetus, respectively. Diagnostic images are obtained from these systems by placing a scan head against the skin of a patient, and actuating an ultrasonic transducer located within the scan head to transmit ultrasonic energy through the skin and into the body of the patient. In response, ultrasonic echoes are reflected from the interior structure of the body, and the returning acoustic echoes are converted into electrical signals by the transducer in the scan head. Ultrasonic therapeutic systems apply ultrasonic emissions to a patient to produce a therapeutic effect, such as a cosmetic therapeutic effect involving the destruction an adipose tissue as described in U.S. Pat. No. 7,993,289, entitled “Systems and Methods for the Destruction of Adipose Tissue,” the entire disclosure of which is hereby incorporated by reference herein.

For an ultrasound system, the ideal impedance of a source (system based pulser and receiver) used to drive an ultrasound transducer depends on the impedance characteristics of the ultrasound transducer, with a resulting phase angle of zero degrees being ideal. The impedance values of ultrasound transducers, however, vary from unit to unit to some degree. Existing approaches for tuning a source to a transducer can be inconvenient (e.g., labor intensive). Additionally, high intensity ultrasound systems, such as used in some ultrasonic therapeutic systems, use power levels which may, over time, damage existing tuning networks.

Accordingly, improved approaches for inductively tuning an ultrasonic system are desirable. For example, improved approaches for tuning ultrasonic systems to account for the impedance characteristics of the particular ultrasound transducer employed are desirable. And improved tuning assemblies that can withstand prolonged exposure to the high power levels used in high intensity ultrasound systems are desirable.

### BRIEF SUMMARY

The following presents a simplified summary of some embodiments of the invention in order to provide a basic understanding of the invention. This summary is not an extensive overview of the invention. It is not intended to identify key/critical elements of the invention or to delineate the scope of the invention. Its sole purpose is to present some embodiments of the invention in a simplified form as a prelude to the more detailed description that is presented later.

Tuning assemblies and related methods are disclosed for inductively tuning ultrasonic systems. The disclosed tuning assemblies and related methods can be used to in conjunction with ultrasound transducer configurations that exhibit unit to unit variation in impedance characteristics so as to adjust the impedance of the ultrasonic system appropriate to the impedance characteristics of the ultrasound transducer used in a particular ultrasonic system. The disclosed tuning assemblies and related methods provide for fast and convenient tuning of ultrasonic systems. And the disclosed tuning systems and methods may better tolerate the high power levels used to drive high intensity ultrasound transducers as compared to existing tuning networks.

Thus, in one aspect, an adjustable tuning network for a high intensity ultrasonic transducer is provided. The tuning network includes inductors and a multi-position (e.g., 16) rotary switch. The tuning network provides impedance matching for the transducer. The switch is used to vary the amount of inductance placed in parallel with the transducer to minimize phase angle. Tuning is accomplished by coupling the transducer to water and working through the rotary switch positions until the phase angle is closest to zero. The network primarily tunes phase angle.

In another aspect, a method is provided for forming a tuned ultrasound transducer assembly and assembling the tuned ultrasound transducer assembly into a medical device. The method includes selecting a tuning assembly that is adjustable to provide a selected inductance value from at least three different inductance values, electrically connecting an ultrasound transducer controller of the medical device with an input side of a transducer via an input electrical conductor, and electrically connecting the tuning assembly between the input electrical conductor and a ground potential so as to provide the selected inductance between the input electrical conductor and the ground potential. The ultrasound transducer controller and the transducer are electrically coupled with the ground potential. At least one of the at least three different inductance values is appropriate to the operation of the transducer. The transducer is operable to produce a cyclic sound pressure output with a frequency of at least 20 kilohertz. The tuning assembly includes a switch and a plurality of tuning inductors. The switch includes a first connection and a plurality of second connections. The switch has at least three positions, each position being operable to selectively connect the first connection with any one of at least three different combinations of the second connections including a combination connecting the first connection with at least two of the second connections. Each of the second connections is connected to at least one of the tuning inductors so that the switch is operable to select which subset of the tuning inductors is connected to the first connection so as to provide the selected inductance value. The switch can include a position selection feature that is rotated to select one of the positions.

In many embodiments, the tuning assembly is adjustable to provide the selected inductance value from at least six different inductance values, at least one of the six different inductance values being appropriate to the operation of the transducer. And the switch can include at least three of the second connections and have at least six positions, each of the six positions being operable to selectively connect the first connection with any one of at least six different combinations of the second connections.

In many embodiments, the tuning assembly is adjustable to provide the selected inductance value from at least ten different inductance values, at least one of the ten different inductance values being appropriate to the operation of the transducer. And the switch can have four to six of the second connections and have at least ten positions, each of the ten positions being operable to selectively connect the first connection with any one of at least ten different combinations of the second connections.

In many embodiments, the tuning assembly is adjustable to provide the selected inductance value from at least twelve different inductance values, at least one of the twelve different inductance values being appropriate to the operation of the transducer. And the switch can have four or five of the second connections and have at least twelve positions, each of the twelve positions being operable to selectively connect the first connection with any one of at least twelve different combinations of the second connections.

In many embodiments, the tuning assembly is adjustable to provide the selected inductance value from at least sixteen different inductance values, at least one of the sixteen different inductance values being appropriate to the operation of the transducer. And the switch can include at least four of the second connections and have at least sixteen positions, each of the sixteen positions being operable to selectively connect the first connection with any one of at least sixteen different combinations of the second connections.

The method can further include additional acts relating to the selection of a position for the tuning assembly switch. For example, the method can include: (a) measuring a phase angle of the ultrasound transducer assembly with the switch in a selected position; (b) operating the switch to change the switch to a different selected position to increase or decrease the selected inductance in response to whether the measured phase angle is positive or negative; (c) subsequent to act (b), re-measuring the phase angle of the ultrasound transducer assembly; and (d) repeating acts (b) and (c) until a change in sign of the measured phase angles occurs. And the method can include selecting a position for the switch that minimizes an absolute magnitude of the corresponding measured phase angle.

The method can include electrically connecting the tuning assembly to a printed circuit board (PCB) and electrically connecting the PCB between the input electrical conductor and the ground potential. The PCB can have a first lead and a second lead so that the PCB first lead and the tuning assembly switch first connection are electrically connected and the PCB second lead is electrically connected with the tuning inductors, each of the tuning inductors being electrically connected between the PCB second lead and a corresponding connection of the switch second connections. The PCB first lead and the input electrical conductor can be electrically connected and the PCB second lead and the ground potential can be electrically connected. Alternatively, the PCB first lead and the ground potential can be electrically connected and the PCB second lead and the input electrical conductor can be electrically connected. The PCB can be coupled to a housing. And the transducer can be secured to the housing so that a fluid tight seal is formed between the transducer and the housing.

In many embodiments, the transducer includes a high intensity focused ultrasound (HIFU) transducer operable to produce ultrasound output having a power level of at least 1 Watt average and typically higher than 10 Watts average to achieve a therapeutic effect in a timely manner. Peak power levels can range from the average power to many multiples of the average power to, for example, take advantage of non-linear effects such as associated heating. And the tuning assembly can include an inductor having an open core construction, which may better tolerate the power levels used to operate the HIFU transducer.

In many embodiments, the method further includes electrically connecting a plurality of fixed inductors in parallel between the input electrical conductor and the ground potential to provide a fixed inductance value between the input electrical conductor and the ground potential. The fixed inductance value and the selectable inductance values can have suitable values. For example, in many embodiments the fixed inductance value can be between 2.0 uH and 7.0 uH and the range of selectable inductance values can be within a range suitable for a particular transducer configuration, for example, between 0.15 uH and 150 uH.

In another aspect, a medical device having a tunable ultrasound transducer assembly is provided. The medical device includes an ultrasound transducer controller electrically connected with a ground potential, a transducer having an input side connected with the ultrasound transducer controller via an input electrical conductor, and a tuning assembly that is electrically connected between the input electrical conductor and the ground potential. The transducer is operable to produce a cyclic sound pressure output with a frequency of at least 20 kilohertz. The transducer is electrically connected with the ground potential. The tuning assembly is adjustable to provide a selected inductance from at least three different inductance values, at least one of the at least three different inductance values being appropriate to the operation of the transducer. The tuning assembly includes a switch and a plurality of tuning inductors. The switch includes a first connection and a plurality of second connections. The switch has at least three positions, each position being operable to selectively connect the first connection with any one of at least three different combinations of the second connections including a combination connecting the first connection with at least two of the second connections. Each of the second connections is connected to at least one of the tuning inductors so that the switch is operable to select which subset of the tuning inductors is connected to the first connection so as to provide the selected inductance value. The switch can include a position selection feature that is rotated to select one of the positions.

In many embodiments, the medical device tuning assembly is adjustable to provide the selected inductance value from at least six different inductance values, at least one of the six different inductance values being appropriate to the operation of the transducer. And the switch can include at least three of the second connections and have at least six positions, each of the six positions being operable to selectively connect the first connection with any one of at least six different combinations of the second connections.

In many embodiments, the medical device tuning assembly is adjustable to provide the selected inductance value from at least ten different inductance values, at least one of the ten different inductance values being appropriate to the operation of the transducer. And the switch can have four to six of the second connections and have at least ten positions, each of the ten positions being operable to selectively connect the first connection with any one of at least ten different combinations of the second connections.

In many embodiments, the medical device tuning assembly is adjustable to provide the selected inductance value from at least twelve different inductance values, at least one of the twelve different inductance values being appropriate to the operation of the transducer. And the switch can have four or five of the second connections and have at least twelve positions, each of the twelve positions being operable to selectively connect the first connection with any one of at least twelve different combinations of the second connections.

In many embodiments, the medical device tuning assembly is adjustable to provide the selected inductance value from at least sixteen different inductance values, at least one of the sixteen different inductance values being appropriate to the operation of the transducer. And the switch can include at least four of the second connections and have at least sixteen positions, each of the sixteen positions being operable to selectively connect the first connection with any one of at least sixteen different combinations of the second connections.

The medical device can include a housing and a printed circuit board (PCB) coupled with the housing. In many embodiments, the transducer is secured to the housing so that a fluid tight seal is formed between the transducer and the housing. The PCB can have a first lead and a second lead so that the PCB first lead and the tuning assembly switch first connection are electrically connected and the PCB second lead is electrically connected with the tuning inductors, each of the tuning inductors being electrically connected between the PCB second lead and a corresponding connection of the switch second connections. The PCB first lead and the input electrical conductor can be electrically connected and the PCB second lead and the ground potential can be electrically connected. Alternatively, the PCB first lead and the ground potential can be electrically connected and the PCB second lead and the input electrical conductor can be electrically connected.

In many embodiments, the medical device transducer includes a high intensity focused ultrasound (HIFU) transducer operable to produce ultrasound output having a power level of at least 1 Watt average and typically higher than 10 Watts average to achieve a therapeutic effect in a timely manner. Peak power levels can range from the average power to many multiples of the average power to, for example, take advantage of non-linear effects such as associated heating. And the medical device tuning assembly can include an inductor having an open core construction, which may better tolerate the power levels used to operate the HIFU transducer.

In many embodiments, the medical device includes a plurality of fixed inductors connected in parallel between the input electrical conductor and the ground potential to provide a fixed inductance value between the input electrical conductor and the ground potential. The fixed inductance value and the selected inductance value can have suitable values. For example, in many embodiments the fixed inductance value can be between 2.0 uH and 7.0 uH and the range of selectable inductance values can be within a range suitable for a particular transducer configuration, for example, between 0.15 uH and 150 uH.

For a fuller understanding of the nature and advantages of the present invention, reference should be made to the ensuing detailed description and accompanying drawings.

### BRIEF DESCRIPTION OF THE DRAWINGS

FIG. 1 illustrates how the impedance characteristics of an exemplary ultrasound transducer configuration can vary from unit to unit.

FIG. 2 schematically illustrates a tunable ultrasound transducer assembly, in accordance with many embodiments.

FIG. 3 schematically illustrates a tuning assembly for inductively tuning an ultrasonic system, in accordance with many embodiments.

FIG. 4 shows a sectional view illustrating a tunable ultrasound transducer assembly, in accordance with many embodiments.

FIG. 5 shows acts of a method for forming a tuned ultrasound transducer assembly, in accordance with many embodiments.

FIG. 6 shows optional acts that can be used in conjunction with the method of FIG. 5 to tune the ultrasound transducer assembly, in accordance with many embodiments.

FIG. 7 shows additional optional acts that can be used in conjunction with the method of FIG. 5, in accordance with many embodiments.

### DETAILED DESCRIPTION

In the following description, various embodiments of the present invention will be described. For purposes of explanation, specific configurations and details are set forth in order to provide a thorough understanding of the embodiments. However, it will also be apparent to one skilled in the art that the present invention can be practiced without the specific details. Furthermore, well-known features may be omitted or simplified in order not to obscure the embodiment being described.

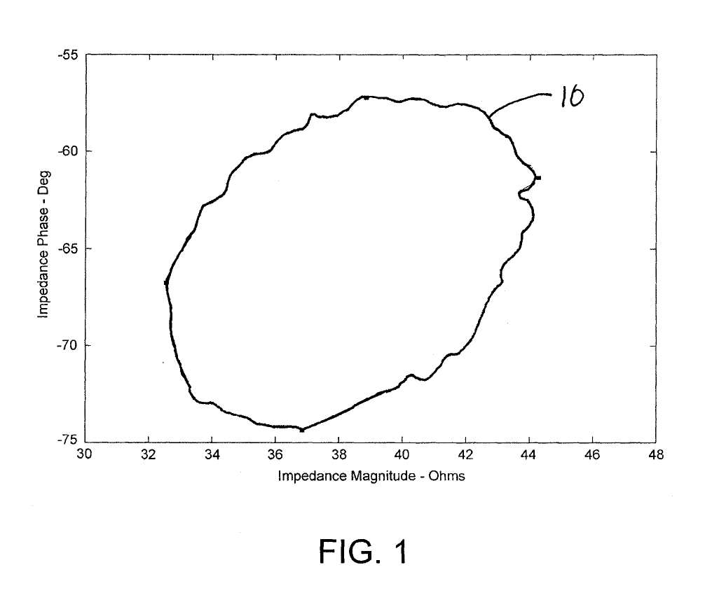

Referring now to the drawings, in which like reference numerals represent like parts throughout the several views, FIG. 1 shows the results of a tolerance analysis of the un-tuned impedance of a high intensity focused ultrasound (HIFU) transducer configuration in the form of a contour 10 in P2 impedance space enclosing a region with 99% probability of occurrence. As illustrated, the unit to unit un-tuned impedance within this 99% probability population can vary significantly, for example, from impedance magnitudes from about 32.5 Ohms to about 44 Ohms and from impedance phase angles of about −57 degrees to about −74 degrees. The HIFU transducer configuration has a center frequency of 2.15 MHz+/−3.2%.

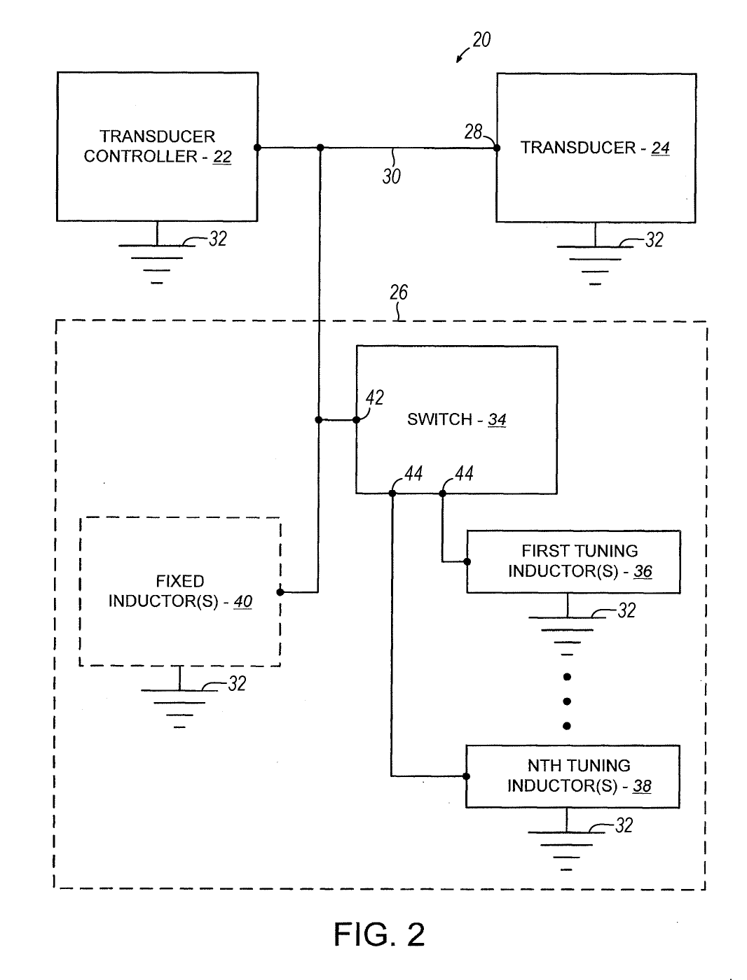

FIG. 2 schematically illustrates a tunable ultrasound transducer assembly 20, in accordance with many embodiments, that includes a transducer controller 22, a transducer 24, and a tuning assembly 26 that can be used to compensate for unit to unit variation in impedance characteristics of the transducer 24, for example, the unit to unit impedance characteristic variations as shown in FIG. 1. An input side of the transducer 28 is electrically coupled with the transducer controller 22 via an input electrical conductor 30. Each of the transducer controller 22 and the transducer 24 are electrically coupled with a ground potential 32, thereby completing a circuit by which the transducer controller 22 drives the transducer 24.

The tuning assembly 26 is adjustable to provide a selected inductance value between the input electrical conductor 30 and the ground potential 32 so that a phase angle of a signal used to drive the transducer 24 can be adjusted to be close to zero degrees. The tuning assembly 26 includes a switch 34 (e.g., a rotary Dual In-Line Package (DIP) switch), a plurality of tuning inductors (one or more first tuning inductors 36 through one or more n^{th} tuning inductors 38), and optionally one or more fixed inductors 40. Each of the one or more first tuning inductors 36 through the one or more n^{th} tuning inductors 38 can include, for example, a single inductor, two or more inductors connected in series, two or more inductors connected in parallel, and any suitable combination of single, series, and/or parallel connected inductors.

The switch 34 includes a first connection 42 and a plurality of second connections 44. The second connections 44 are connected to the one or more first tuning inductor(s) 36 through the one or more n^{th} tuning inductor(s) 38. In many embodiments, the switch 34 has multiple positions including positions that connect the first connection 42 to only one of the second connections 44 and one or more positions that connect the first connection 42 to two or more of the second connections 44, thereby increasing the number of possible inductance values that can be connected to and between the input electrical conductor 30 and the ground potential 32.

Accordingly, the switch 34 is operable to connect a selected combination of the one or more first tuning inductors 36 through the one or more n^{th} tuning inductors 38 to and between the input electrical conductor 30 and the ground potential 32. For example, in many embodiments, the switch 34 has a configuration (i.e., a position) that connects only the first tuning inductor(s) 36 to and between the input electrical conductor 30 and the ground potential 32, additional positions that each connect one of the second through the n^{th} tuning inductors to and between the input electrical conductor 30 and the ground potential 32, and yet one or more additional positions that each connects a different combination of the first through the n^{th} tuning inductors to and between the input electrical conductor 30 and the ground potential 32. In many embodiments, the one or more first tuning inductor(s) 36 through the one or more n^{th} tuning inductor(s) 38 are selected so that each of the one or more first tuning inductor(s) 36 through the one or more n^{th} tuning inductor(s) 32 provides a different inductance value between the switch 34 and the ground potential 32.

In many embodiments, the tuning assembly 26 includes the one or more fixed inductor(s) 40. The combination of the fixed inductance provided by the one or more fixed inductor(s) 40 and the range of selectable inductances provided by the switch 34 and the tuning inductors covers a desired range of total inductances suitable for the expected range of un-tuned transducer impedances for a population of transducers.

The tuning assembly 26 can be configured for use with a transducer operating at a relatively high power level (e.g., greater than 1 Watt average and typically greater than 10 Watts average), such as with a high intensity focused ultrasound (HIFU) transducer used in a therapeutic ultrasound system. For example, in many embodiments, the fixed inductors 40 include two or more inductors connected in parallel so as to reduce the current that any one of the fixed inductors is exposed to, thereby increasing the resulting life of the fixed inductors by reducing the power (and the resulting heat) generated by the fixed inductors. Likewise, the tuning inductors can also be to connected in series or parallel to a suitable extent so as to reduce the voltage or current that any one of the tuning inductors is exposed to, thereby increasing the resulting life of the tuning inductors by reducing the power (and resulting heat) generated by the tuning inductors. And the tuning assembly 26 can use inductors (for the fixed and/or the tuning inductors) having an open-core configuration, which are typically used for large rating power converters and may better tolerate the current levels associated with the operation of a high intensity ultrasound transducer as compared to inductors having a non-open-core configuration.

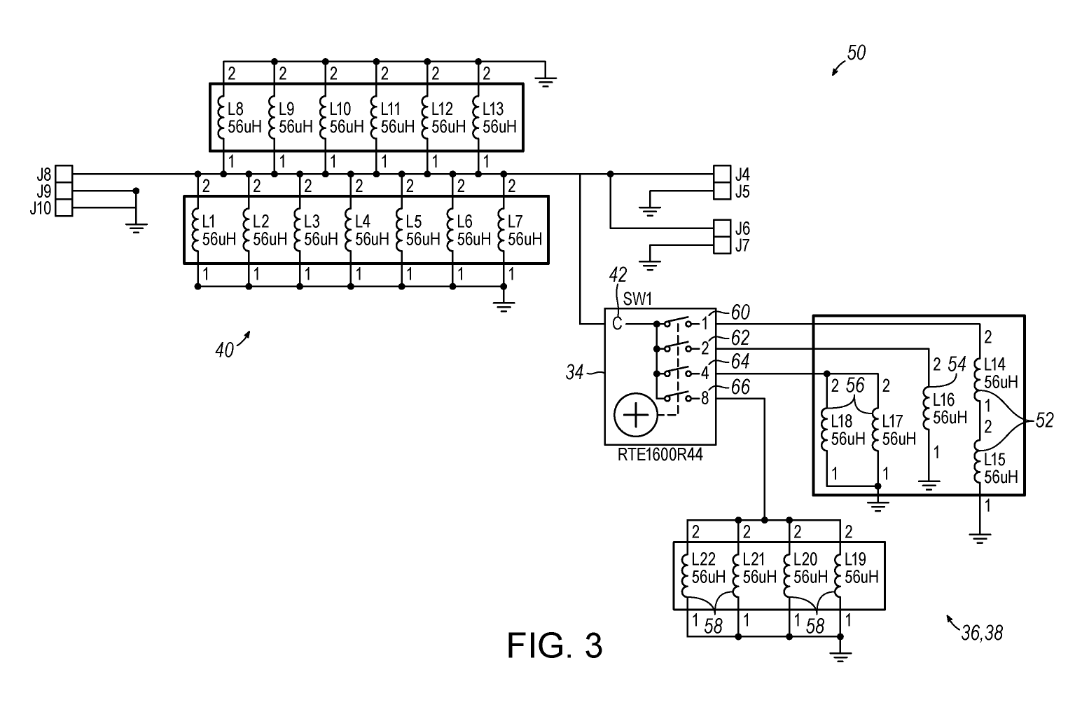

FIG. 3 schematically illustrates a tuning assembly 50 for inductively tuning an ultrasonic system, in accordance with many embodiments. The tuning assembly 50 is constructed in conformance with the above-described tuning assembly 26 of FIG. 2, but includes a specific configuration that provides an example of how the fixed inductor(s) 40, the switch 34, and the tuning inductors 36, 38 can be configured to provide the tuning assembly 50 with excellent adjustability (e.g., 16 different selectable inductances) as well as the capability for use with a high intensity ultrasound transducer system due to the use of open-core inductors connected in parallel.

The fixed inductors 40 include thirteen 56 uH open-core inductors (e.g., Coilcraft part number ME3220-563KLB) connected in parallel. Accordingly, the thirteen fixed inductors provide an equivalent inductance of 4.31 uH. In many embodiments, surface mount inductors are used because they are suitable for automated assembly and are state of the art.

The tuning inductors 36, 38 include nine 56 uH open-core inductors connected in the illustrated ways, including two first tuning inductors 52 connected in series, a single second tuning inductor 54, two third tuning inductors 56 connected in parallel, and four fourth tuning inductors 58 connected in parallel. The two first tuning inductors 52 provide a combined inductance of 112 uH. The two third tuning inductors 56 provide an equivalent inductance of 28 uH. And the four fourth tuning inductors 58 provide an equivalent inductance of 14 uH.

The switch 34 is an RTE1600R44 low profile rotary dual in-line package (DIP) switch having 16 positions with a hexadecimal code switch function. The switch 34 includes a first connection 42 and four second connections 60, 62, 64, 66. Table 1 below illustrates the connections between the first connection 42 and the four second connections 60, 62, 64, 66, as well as the resulting tuning inductance and total inductance, for each of the 16 positions.

TABLE 1
Tuning Inductance and Total Inductance by Switch Position

| | First | Second | Third | Fourth | | |
|---|---|---|---|---|---|---|
| | Tuning | Tuning | Tuning | Tuning | | |
| Switch | Inductors - | Inductor - | Inductors - | Inductors - | Tuning | Total |
| Position | 52 | 54 | 56 | 58 | Inductance | Inductance |
| 0 | open | open | open | open | open | 4.31 uH |
| 1 | 114 uH | open | open | open | 114 uH | 4.15 uH |
| 2 | open | 56 uH | open | open | 56.00 uH | 4.00 uH |
| 3 | 114 uH | 56 uH | open | open | 37.55 uH | 3.86 uH |
| 4 | open | open | 28 uH | open | 28.00 uH | 3.73 uH |
| 5 | 114 uH | open | 28 uH | open | 22.48 uH | 3.61 uH |
| 6 | open | 56 uH | 28 uH | open | 18.67 uH | 3.50 uH |
| 7 | 114 uH | 56 uH | 28 uH | open | 16.04 uH | 3.40 uH |
| 8 | open | open | open | 14 uH | 14.00 uH | 3.29 uH |
| 9 | 114 uH | open | open | 14 uH | 12.47 uH | 3.20 uH |
| 10 | open | 56 uH | open | 14 uH | 11.20 uH | 3.11 uH |
| 11 | 114 uH | 56 uH | open | 14 uH | 10.20 uH | 3.03 uH |
| 12 | open | open | 28 uH | 14 uH | 9.33 uH | 2.95 uH |
| 13 | 114 uH | open | 28 uH | 14 uH | 8.63 uH | 2.87 uH |
| 14 | open | 56 uH | 28 uH | 14 uH | 8.00 uH | 2.80 uH |
| 15 | 114 uH | 56 uH | 28 uH | 14 uH | 7.48 uH | 2.73 uH |

Note:
open = no inductor connected.

As shown in the resulting total inductances listed in Table 1, the tuning assembly 50 is operable to provide any one selected inductance between the input electrical conductor 30 and the ground potential 32 ranging from a low of 2.73 uH with the switch 34 in position 15 to a high of 4.31 uH with the switch 34 in position 0. The tuning assembly 50 is configured to provide a range of tuning suitable for the unit to unit transducer impedance variation exhibited in FIG. 1. For example, the tuned impedance is:

Z_{tuned}=1/((1/Z_{transducer})+(1/Z_{tuneinductor}))

where: Z_{tuneinductor}=j*2*pi*f*L_{tuneinductor}

L_{tuneinductor}=inductance of the tuning inductor

f=operating frequency

Setting the phase angle of Z_{tuned}=0 for ideal tuning and solving for L_{tuneinductor} yields:

L_{tuneinductor}=Magnitude(Z_{transducer})/(2*pi*f*sin(−Phase(Z_{transducer})))

Table 2 below includes the values of transducer impedance, from FIG. 1, at the corners (min/max magnitude and min/max phase angle) along with the ideal tuning inductor value. All values are at f=2 Mhz. As can be seen, the tuning range of Table 1 covers the tuning values of Table 2.

TABLE 2
Example Ideal Tuning Inductor Values

| Z_{transducer} (Ohms) | L_{tuneinductor} (uH) |
|---|---|
| 32.5714 at −66.7460 degrees | 2.82 |
| 38.8751 at −57.2222 degrees | 3.68 |
| 44.2857 at −61.3492 degrees | 4.02 |
| 36.8571 at −74.3651 degrees | 3.05 |

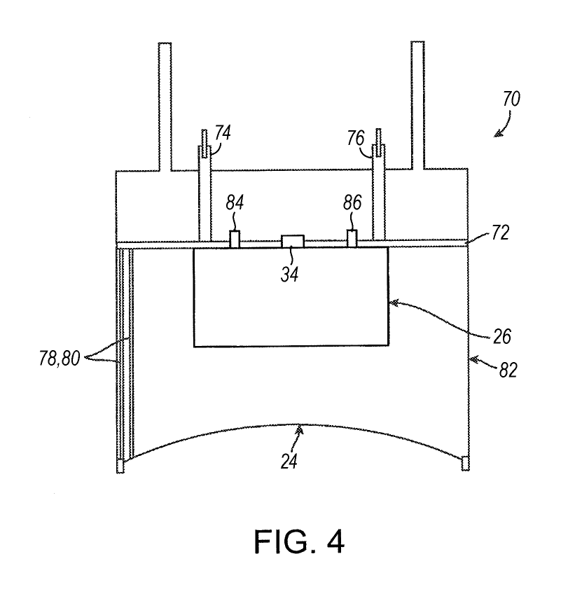

FIG. 4 shows a cross-section of a tunable ultrasound transducer assembly 70, in accordance with many embodiments. Components of the tunable ultrasound transducer assembly 70 common to the above-described tunable ultrasound transducer assembly 20 of FIG. 2 share the same reference numerals. The tunable ultrasound transducer assembly 70 includes the tuning assembly 26, a printed circuit board (PCB) 72, the transducer 24, a system connection first electrical path 74, a system connection second electrical path 76, a transducer connection first electrical path 78, a transducer connection second electrical path 80, and a housing 82.

As in the tunable ultrasound transducer assembly 20 of FIG. 2, the tuning assembly 26 provides a selectable impedance between the ground potential 32 and an electrical path used to transfer a driving signal from the transducer controller 22 to the transducer 24. The system connection first electrical path 74 is configured to be electrically connected with the transducer controller 22 to receive the driving signal. The system connection second electrical path 76 is configured to be electrically connected with the ground potential 32. The system connection first and second electrical paths 74, 76 are shown as a pair of spring loaded connection pins on “top” of the PCB 72 and are configured to engage corresponding electrical traces in a receptacle that is electrically coupled with the transducer controller 22 and the ground potential 32. The PCB 72 includes a first electrical trace (not shown) that electrically couples the system connection first electrical path 74 to the transducer connection first electrical path 78. And the PCB includes a second electrical trace (not shown) that electrically couples the system connection second electrical path 76 to the transducer connection second electrical path 80. The tuning assembly 26 includes a first connection 84 that is electrically coupled with the PCB first electrical trace and a second connection 86 that is electrically coupled with the PCB second electrical trace, thereby electrically coupling the tuning assembly 26 between the driving signal in the PCB first electrical trace and the ground potential 32 that is electrically coupled with the PCB second electrical trace. The tuning assembly 26 includes the switch 34, which is selectively operated as described herein to select the impedance provided by the tuning assembly 26.

Tuned Ultrasound Transducer Assembly Methods

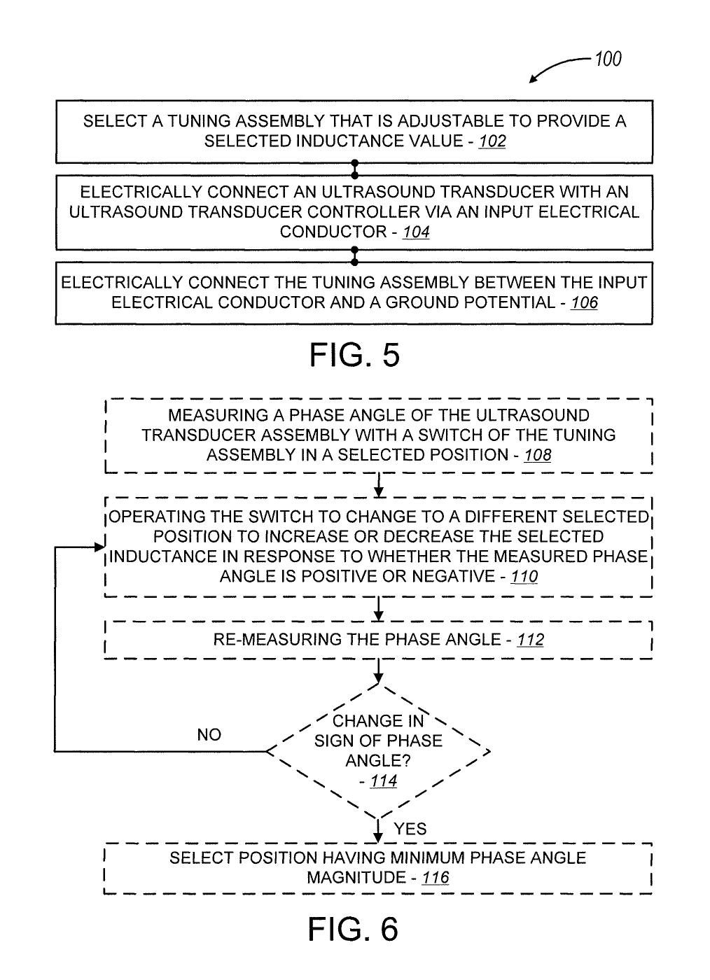

FIG. 5 shows acts of a method 100 for forming a tuned ultrasound transducer assembly and assembling the tuned ultrasound transducer assembly into a medical device, in accordance with many embodiments. The assemblies and components described herein can be used to practice the method 100. In act 102, a tuning assembly (e.g., tuning assembly 26, 50) is selected that is adjustable to provide a selected inductance value. In many embodiments, the tuning assembly is adjustable to provide the selected inductance value from a number of different inductance values as described herein. In act 104, an ultrasound transducer (e.g., the ultrasound transducer 24) is electrically connected with an ultrasound transducer controller (e.g., the ultrasound controller 22) via an input electrical conductor (e.g., the input electrical conductor 30). And in act 106, the tuning assembly is electrically connected between the input electrical conductor and a ground potential.

FIG. 6 shows optional acts that can be used in conjunction with the method 100 to tune the ultrasound transducer assembly, in accordance with many embodiments. In act 108, with the switch of the tuning assembly in a selected position, a phase angle of the ultrasound transducer assembly is measured. In act 110, the switch is operated to change to a different selected position to increase or decrease the selected inductance in response to whether the measured phase angle is positive or negative. For example, if the phase angle measurement is a positive 15 degrees, the switch can be operated in a direction so that the newly selected inductance will result in a reduction of the measured phase angle corresponding to the newly selected inductance. In act 112, the phase angle of the ultrasound transducer assembly is re-measured, thereby providing a phase angle measurement corresponding to the newly selected inductance. In act 114, sequential phase angle measurements are inspected to see if a change in sign of the phase angle measurements has occurred. If no change in sign of the phase angle measurements has occurred, act 110 through act 114 are repeated until a change in sign of the phase angle measurements has occurred. Once a change in sign in the phase angle measurements has occurred, the switch position providing the minimum phase angle magnitude can be selected in act 116.

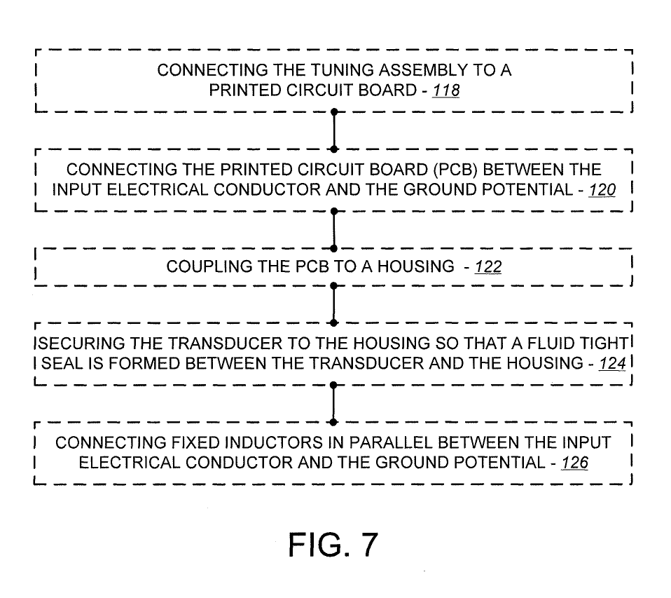

FIG. 7 shows additional optional acts that can be used in conjunction with the method 100, in accordance with many embodiments. In act 118, the tuning assembly is connected to a printed circuit board (PCB) (e.g., the PCB 72). In many embodiments, the tuning assembly is both physically attached to the PCB and electrically connected to the PCB as in the tunable ultrasound transducer assembly 70 of FIG. 4. In act 120, the PCB is electrically connected between the input electrical conductor and the ground potential. In act 122, the PCB is coupled to a housing (e.g., the housing 82). In act 124, the transducer is secured to the housing so that a fluid tight seal is formed between the transducer and the housing. And in act 126, fixed inductors (e.g., the fixed inductor(s) 140) are connected in parallel between the input electrical conductor and the ground potential.

Other variations are within the spirit of the present invention. Thus, while the invention is susceptible to various modifications and alternative constructions, certain illustrated embodiments thereof are shown in the drawings and have been described above in detail. It should be understood, however, that there is no intention to limit the invention to the specific form or forms disclosed, but on the contrary, the intention is to cover all modifications, alternative constructions, and equivalents falling within the spirit and scope of the invention, as defined in the appended claims.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. The term “connected” is to be construed as partly or wholly contained within, attached to, or joined together, even if there is something intervening. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate embodiments of the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

All references, including publications, patent applications, and patents, cited herein are hereby incorporated by reference to the same extent as if each reference were individually and specifically indicated to be incorporated by reference and were set forth in its entirety herein.

What is claimed is:

1. A method for forming a tuned ultrasound transducer assembly and assembling the tuned ultrasound transducer assembly into a medical device, the medical device having an ultrasound transducer controller, the method comprising:
selecting a tuning assembly that is adjustable to provide a selected inductance value from at least three different inductance values, at least one of the at least three different inductance values being appropriate to the operation of a transducer, the transducer having an input side, the transducer being operable to produce a cyclic sound pressure output with a frequency of at least 20 kilohertz, the tuning assembly including a switch and a plurality of tuning inductors, the switch including a first connection and a plurality of second connections, the switch having at least three positions, each position being operable to selectively connect the first connection with any one of at least three different combinations of the second connections including a combination connecting the first connection to at least two of the second connections, each of the second connections being connected to at least one of the tuning inductors so that the switch is operable to select which subset of the tuning inductors is connected to the first connection so as to provide the selected inductance value;
electrically connecting the ultrasound transducer controller with the transducer input side via an input electrical conductor; and
electrically connecting the tuning assembly between the input electrical conductor and a ground potential so as to provide the selected inductance between the input electrical conductor and the ground potential, the ultrasound transducer controller and the transducer being electrically coupled with the ground potential.

2. The method of claim 1, wherein:
the tuning assembly is adjustable to provide the selected inductance value from at least six different inductance values, at least one of the at least six different inductance values being appropriate to the operation of the transducer;
the switch includes at least three of the second connections; and
the switch has at least six positions, each position being operable to selectively connect the first connection with any one of at least six different combinations of the second connections.

3. The method of claim 2, wherein:
the tuning assembly is adjustable to provide the selected inductance value from at least ten different inductance values, at least one of the at least ten different inductance values being appropriate to the operation of the transducer;
the switch has four to six of the second connections; and
the switch has at least ten positions, each position being operable to selectively connect the first connection with any one of at least ten different combinations of the second connections.

4. The method of claim 3, wherein:
the tuning assembly is adjustable to provide the selected inductance value from at least twelve different inductance values, at least one of the at least twelve different inductance values being appropriate to the operation of the transducer;
the switch has four or five of the second connections; and
the switch has at least twelve positions, each position being operable to selectively connect the first connection with any one of at least twelve different combinations of the second connections.

5. The method of claim 2, wherein:
the tuning assembly is adjustable to provide the selected inductance value from at least sixteen different inductance values, at least one of the at least sixteen different inductance values being appropriate to the operation of the transducer;
the switch includes at least four of the second connections; and
the switch has at least sixteen positions, each position being operable to selectively connect the first connection with any one of at least sixteen different combinations of the second connections.

6. The method of claim 5, wherein the switch includes a position selection feature that is rotated to select one of the at least sixteen positions.

7. The method of claim 4, wherein the switch includes a position selection feature that is rotated to select one of the at least twelve positions.

8. The method of claim 3, wherein the switch includes a position selection feature that is rotated to select one of the at least ten positions.

9. The method of claim 2, wherein the switch includes a position selection feature that is rotated to select one of the at least six positions.

10. The method of claim 9, further comprising:
(a) measuring a phase angle of the ultrasound transducer assembly with the switch in a selected position of the at least six positions;
(b) operating the switch to change the switch to a different selected position to increase or decrease the selected inductance in response to whether the measured phase angle is positive or negative;
(c) subsequent to act (b), re-measuring the phase angle of the ultrasound transducer assembly; and
(d) repeating acts (b) and (c) until a change in sign of the measured phase angles occurs.

11. The method of claim 10, further comprising selecting a position for the switch from the at least six positions that minimizes an absolute magnitude of the corresponding measured phase angle.

12. The method of claim 1, wherein the switch includes a position selection feature that is rotated to select one of the at least three positions.

13. The method of claim 1, further comprising:
electrically connecting the tuning assembly to a printed circuit board (PCB) having a first lead and a second lead so that the PCB first lead and the tuning assembly switch first connection are electrically connected and the PCB second lead is electrically connected with the tuning inductors, each of the tuning inductors being electrically connected between the PCB second lead and corresponding connections of the switch second connections; and
electrically connecting the PCB between the input electrical conductor and the ground potential so that (1) the PCB first lead and the input electrical conductor are electrically connected and the PCB second lead and the ground potential are electrically connected, or (2) the PCB first lead and the ground potential are electrically connected and the PCB second lead and the input electrical conductor are electrically connected.

14. The method of claim 13, further comprising:
coupling the PCB to a housing; and
securing the transducer to the housing so that a fluid tight seal is formed between the transducer and the housing.

15. The method of claim 1, wherein the transducer comprises a high intensity focused ultrasound (HIFU) transducer operable to produce an ultrasound output having an average power of at least 1 Watt.

16. The method of claim 15, wherein the ultrasound output has an average power of at least 10 Watts.

17. The method of claim 15, wherein the tuning assembly inductors comprise an inductor having an open core construction.

18. The method of claim 1, further comprising electrically connecting a plurality of fixed inductors in parallel between the input electrical conductor and the ground potential to provide a fixed inductance value between the input electrical conductor and the ground potential.

19. The method of claim 18, wherein the fixed inductance value is between 2.0 uH and 7.0 uH and the selected inductance value is adjustable within a range that lies between 0.15 uH and 150 uH.

20. A medical device having a tunable ultrasound transducer assembly, the medical device comprising:
an ultrasound transducer controller electrically connected with a ground potential;
a transducer having an input side connected with the ultrasound transducer controller via an input electrical conductor, the transducer being operable to produce a cyclic sound pressure output with a frequency of at least 20 kilohertz, the transducer being electrically connected with the ground potential; and
a tuning assembly that is electrically connected between the input electrical conductor and the ground potential, the tuning assembly being adjustable to provide a selected inductance value from at least three different inductance values, at least one of the at least three different inductance values being appropriate to the operation of the transducer, the tuning assembly including a switch and a plurality of tuning inductors, the switch including a first connection and a plurality of second connections, the switch having at least three positions, each position being operable to selectively connect the first connection with any one of at least three different combinations of the second connections including a combination connecting the first connection to at least two of the second connections, each of the second connections being connected to at least one of the tuning inductors so that the switch is operable to select which subset of the tuning inductors is connected to the first connection so as to provide the selected inductance value.

21. The device of claim 20, wherein:
the tuning assembly is adjustable to provide the selected inductance value from at least six different inductance values, at least one of the at least six different inductance values being appropriate to the operation of the transducer;
the switch includes at least three of the second connections; and
the switch has at least six positions, each position being operable to selectively connect the first connection with any one of at least six different combinations of the second connections.

22. The device of claim 21, wherein:
the tuning assembly is adjustable to provide the selected inductance value from at least ten different inductance values, at least one of the at least ten different inductance values being appropriate to the operation of the transducer;
the switch has four to six of the second connections; and
the switch has at least ten positions, each position being operable to selectively connect the first connection with any one of at least ten different combinations of the second connections.

23. The device of claim 22, wherein:
the tuning assembly is adjustable to provide the selected inductance value from at least twelve different inductance values, at least one of the at least twelve different inductance values being appropriate to the operation of the transducer;
the switch has four or five of the second connections; and
the switch has at least twelve positions, each position being operable to selectively connect the first connection with any one of at least twelve different combinations of the second connections.

24. The device of claim 21, wherein:
the tuning assembly is adjustable to provide the selected inductance value from at least sixteen different inductance values, at least one of the at least sixteen different inductance values being appropriate to the operation of the transducer;
the switch includes at least four of the second connections; and
the switch has at least sixteen positions, each position being operable to selectively connect the first connection with any one of at least sixteen different combinations of the second connections.

25. The device of claim 24, wherein the switch includes a position selection feature that is rotated to select one of the at least sixteen positions.

26. The device of claim 23, wherein the switch includes a position selection feature that is rotated to select one of the at least twelve positions.

27. The device of claim 22, wherein the switch includes a position selection feature that is rotated to select one of the at least ten positions.

28. The device of claim 21, wherein the switch includes a position selection feature that is rotated to select one of the at least six positions.

29. The device of claim 20, wherein the switch includes a position selection feature that is rotated to select one of the at least three positions.

30. The device of claim 20, further comprising:
a housing, the transducer being secured to the housing so that a fluid tight seal is formed between the transducer and the housing; and
a printed circuit board (PCB) coupled with the housing, the PCB having a first lead and a second lead so that the PCB first lead and the tuning assembly switch first connection are electrically connected and the PCB second lead is electrically connected with the tuning inductors, each of the tuning inductors being electrically connected between the PCB second lead and corresponding connections of the switch second connections, the PCB being electrically connected between the input electrical conductor and the ground potential so that (1) the PCB first lead and the input electrical conductor are electrically connected and the PCB second lead and the ground potential are electrically connected, or (2) the PCB first lead and the ground potential are electrically connected and the PCB second lead and the input electrical conductor are electrically connected.

31. The device of claim 20, wherein the transducer comprises a high intensity focused ultrasound (HIFU) transducer operable to produce an ultrasound output having an average power of at least 1 Watt.

32. The device of claim 31, wherein the ultrasound output has an average power of at least 10 Watts.

33. The device of claim 31, wherein the tuning inductors comprise an inductor having an open core construction.

34. The device of claim 20, further comprising a plurality of fixed inductors connected in parallel between the input electrical conductor and the ground potential to provide a fixed inductance value between the input electrical conductor and the ground potential.

35. The device of claim 34, wherein the fixed inductance value is between 2.0 uH and 7.0 uH and the selected inductance value is adjustable within a range that lies between 0.15 uH and 150 uH.
